(19) United States
(12) Patent Application Publication
(10) Pub. No.: US 2022/0259127 A1
(43) Pub. Date: Aug. 18, 2022

(54) METHOD AND FACILITY FOR PRODUCING A TARGET COMPOUND

(71) Applicant: LINDE GMBH, Pullach (DE)

(72) Inventors: Andreas MEISWINKEL, Rimsting (DE); Hans-Jörg ZANDER, München (DE); Ernst HAIDEGGER, Riemerling (DE); Isabel KIENDL, München (DE)

(21) Appl. No.: 17/627,803

(22) PCT Filed: Jul. 16, 2020
(86) PCT No.: PCT/EP2020/070197

§ 371 Date: Jan. 17, 2022

(30) Foreign Application Priority Data
Jul. 18, 2019 (DE) 10 2019 119 562.4

(51) Int. Cl. C07C 45/50; C07C 29/141; C07C 1/24; C01B 3/16; C07C 2/82

(52) U.S. Cl. CPC C07C 45/50; C07C 29/141; C07C 1/24; C01B 2203/06; C07C 2/82; C01B 2203/0288; C01B 2203/062; C01B 3/16

(57) ABSTRACT

Disclosed is a method for producing a target compound, in which a first gas mixture includes an olefin having a first carbon number and carbon monoxide, a second gas mixture formed using the first gas mixture and containing the olefin, hydrogen and carbon monoxide, is subjected to conversion steps to obtain a third gas mixture containing a compound with a second carbon number and at least carbon monoxide The conversion includes hydroformylation. The second carbon number is one greater than the first carbon number. Using at least a portion of the third gas mixture, a fourth gas mixture which is depleted in the compound has three carbon atoms, is enriched in carbon monoxide, and is formed using at least a portion of the third gas mixture The carbon monoxide in at least a portion of the fourth gas mixture is subjected to a water gas shift to form hydrogen and carbon dioxide, and that the hydrogen formed in the water gas shift is used in the formation of the second gas mixture.

### RELATED APPLICATIONS

The present application is a national stage application under 35 U.S.C. § 371 of International Application No. PCT/EP2020/070191, filed 16 Jul. 2020, which claims priority to German Patent Application No. 10 2019 119 540.3, filed 18 Jul. 2019. The above referenced applications are hereby incorporated by reference in their entirety.

### BACKGROUND

The present invention relates to a method for producing a target compound, in particular propylene, and to a corresponding installation in accordance with the preambles of the independent claims.

The project that has led to the present patent application was promoted within the framework of the financial aid agreement no. 814557 of the European Union's Horizon 2020 Research and Innovations program.

### PRIOR ART

The production of propylene (propene) is described in the specialist literature, for example in the article “Propylene” in Ullmann's Encyclopedia of Industrial Chemistry, ed. 2012. Propylene is conventionally produced by steam cracking hydrocarbon feeds and conversion processes in the course of refinery processes. In the latter processes, propylene is not necessarily formed in the desired amount and only as one of several components in a mixture with further compounds. Other processes for producing propylene are also known, but are not satisfactory in all cases, for example in terms of efficiency and yield.

An increasing demand for propylene (“propylene gap”), which requires the provision of corresponding selective methods, is predicted for the future. At the same time, it is necessary to reduce or even prevent carbon dioxide emissions. As a potential feedstock, on the other hand, large amounts of methane are available, which are currently only fed to a material utilization in a very limited manner and are predominantly burned.

WO 2018/005074 A1 provides methods for the preparation of propanal in a reaction comprising the oxidative coupling of methane and oxygen as reactant stream in a gas phase reaction, preferably in the presence of water or steam, to form ethylene, ethane, carbon dioxide, water and synthesis gas in a first reactor as ethylene stream, and then to form propanal in a second reactor by feeding the ethylene stream with the synthesis gas from the first reactor in the gas phase into the second reactor and hydroformylating in the presence of a catalyst for a water gas shift reaction. In the method, the ratio of hydrogen to carbon monoxide in the synthesis gas is maintained by either feeding steam into the first reactor or into the second reactor to produce additional hydrogen in the synthesis gas, or by forming carbon monoxide from the water gas shift reaction in the second reactor by feeding the carbon dioxide from the ethylene stream into the second reactor.

U.S. Pat. No. 2,464,916 A proposes a method for converting an olefin into an alcohol by carbonylation of the olefin with carbon monoxide and hydrogen at elevated temperature and elevated pressure in the presence of a catalyst containing a metal selected from the group consisting of cobalt and iron, and subsequent hydrogenation of the resulting aldehyde. Here, steps are provided which consist in the fact that the carbonylation is carried out in a first zone with a mixture of hydrogen and carbon monoxide which contains an excess of hydrogen, a liquid carbonylation product and a gaseous stream containing the excess hydrogen and unreacted carbon monoxide are drawn off from the first zone, the gaseous stream in a second zone is brought into contact with an additional amount of the catalyst and a feed material containing the olefin under temperature and pressure conditions at which a carbonyl derivative of the catalyst is formed, a mixture of the feed material and the carbonyl derivative is drawn off from the second zone, and the mixture is fed into the second zone into the first zone as a liquid feed stream to the latter, and drawing off a purified hydrogen stream is drawn off from the second zone, and a hydrogenation of the carbonylation product is carried out thereby.

U.S. Pat. No. 9,856,198 B1 discloses integrated methods for the use of hydroformylation reaction strategies for efficient conversion of ethylene into ethylene feed material mixtures in C3 products (i.e. products comprising 3 carbon atoms), such as propionaldehyde, 1-propanol, propylene, propanoic acid and the like. One aspect is to partially purify a feed comprising an ethane/ethylene mixture, rather than attempting a more complete purification. In contrast to an essentially complete purification of ethylene, partial purification is to be technically and economically feasible and more cost-effective and enables hydroformylation with high productivity and lower hydrogen and carbon monoxide requirements. Since smaller amounts of such synthesis gas are used in the hydroformylation reaction and the reactants remaining in the subsequent hydroformylation have a more favorable profile, recycling strategies are to be able to be applied much more easily.

The object of the present invention is to provide a method for the production of propylene, which is improved in particular in view of these aspects, but also for the production of other organic target compounds, in particular of oxo compounds, such as aldehydes and alcohols with a corresponding carbon backbone.

### DISCLOSURE OF THE INVENTION

Against this background, the present invention proposes a method for producing a target compound, in particular propylene, and a corresponding installation with the respective features of the independent patent claims. Preferred embodiments of the present invention are the subject matter of the dependent claims and of the following description.

In principle, in addition to the aforementioned steam cracking processes, a plurality of different methods exist for converting hydrocarbons and related compounds into one another, some of which will be mentioned below by way of example.

For example, the conversion of paraffins to olefins of identical chain length by oxidative dehydrogenation (ODH, also referred to as ODHE in the case of ethane) is known. The production of propylene from propane by dehydrogenation (PDH) is also known and represents a commercially available and established method. The same also applies to the production of propylene from ethylene by olefin metathesis. This method requires 2-butene as an additional reagent.

Lastly, so-called methane-to-olefin or methane-to-propylene (MTO, MTP) processes exist in which synthesis gas is first produced from methane and the synthesis gas is then converted to give olefins, such as ethylene and propylene. Corresponding processes can be operated on the basis of methane, but also on the basis of other hydrocarbons or carbon-containing starting materials, such as coal or biomass.

However, ethylene can also be produced by the oxidative coupling of methane (OCM). Since the oxidative coupling of methane is used the present invention, it will be explained in more detail below. The oxidative coupling of methane is described in the literature, for example in J. D. Idol et al., “Natural Gas” in: J. A. Kent (ed.), “Handbook of Industrial Chemistry and Biotechnology”, Volume 2, 12th Edition, Springer, New York 2012. In principle, however, in designs not according to the invention, a processing of other gas mixtures, that is to say not provided by the oxidative coupling, is also possible and advantageous when these gas mixtures contain one or more olefins in a significant content, for example more than 10, 20, 30, 40 or 50 mol percent and up to 80 mol percent (as single or total value) and carbon monoxide in such quantity ranges.

According to the current state of knowledge, the oxidative coupling of methane comprises a catalyzed gas phase reaction of methane with oxygen, in which a hydrogen atom is separated from each of two methane molecules. Oxygen and methane are activated on the catalyst surface. The resulting methyl radicals first react to give an ethane molecule. In the reaction, a water molecule is further formed. In the case of suitable ratios of methane to oxygen, suitable reaction temperatures and the choice of suitable catalysis conditions, an oxydehydrogenation of the ethane to ethylene is subsequently effected, a target compound in the oxidative coupling of methane. Here, a further water molecule is formed. The oxygen used is typically converted completely in the aforementioned reactions.

The reaction conditions in the oxidative coupling of methane conventionally include a temperature of 500 to 900° C., a pressure of 5 to 10 bar and high space velocities. More recent developments are also in particular oriented towards the use of lower temperatures. The reaction can take place homogeneously and heterogeneously in the fixed bed or in the fluidized bed. In the oxidative coupling of methane, it is also possible to form higher hydrocarbons having up to six or eight carbon atoms, although the focus is on ethane or ethylene and optionally also propane or propylene.

In particular due to the high binding energy between carbon and hydrogen in the methane molecule, the yields in the oxidative coupling of methane are comparatively low. Typically, no more than 10 to 15% of the methane used is converted. In addition, the comparatively harsh reaction conditions and temperatures which are required for the cleavage of these bonds also promote the further oxidation of the methyl radicals and other intermediates to give carbon monoxide and carbon dioxide. In particular, the use of oxygen plays a dual role here. Thus, the methane conversion is dependent on the oxygen concentration in the mixture. The formation of by-products is coupled to the reaction temperature, since the total oxidation of methane, ethane and ethylene is preferably carried out at high temperatures.

Although the low yields and the formation of carbon monoxide and carbon dioxide can be counteracted partly by the choice of optimized catalysts and adapted reaction conditions, a gas mixture formed in the oxidative coupling of methane contains predominantly unconverted methane and carbon dioxide, carbon monoxide and water besides the target compounds, such as ethylene and optionally propylene. Any non-catalytic cleavage reactions may also contain considerable amounts of hydrogen. In the terminology used here, such a gas mixture is also referred to as “product mixture” of the oxidative coupling of methane, although it predominantly does not contain the desired products, but rather the unconverted reactant methane and the by-products just outlined as well.

In the oxidative coupling of methane, reactors can be used in which a catalytic zone is connected downstream of a non-catalytic zone. The gas mixture flowing out of the catalytic zone is transferred into the non-catalytic zone, where it is initially still present at the comparatively high temperatures which are used in the catalytic zone. In particular, due to the presence of the water formed in the oxidative coupling of methane, the reaction conditions are similar here to those of conventional steam cracking processes. Therefore, ethane and higher paraffins can be converted to olefins here. Further paraffins can also be fed into the non-catalytic zone, so that the residual heat of the oxidative coupling of methane can be utilized in a particularly advantageous manner.

Such targeted steam cracking in a non-catalytic zone downstream of the catalytic zone is also referred to as “post bed cracking”. The term “post-catalytic steam cracking” is also used for this below. If it is stated below that a starting gas mixture used according to the invention is formed or provided by “using” or “exploiting” an oxidative coupling of methane, this specification is not intended to be understood in such a way that only the oxidative coupling itself needs to be used during the provision. Rather, the provision of the starting gas mixture can also comprise further process steps, in particular post-catalytic steam cracking.

According to particularly preferred embodiments of the present invention, paraffins, in particular ethane, which can be separated from any streams at a suitable point or can be contained in corresponding streams, can be recycled alone or together with further components for post-catalytic steam cracking. The separation, if conducted, is carried out at a place suitable for separation, i.e. at a position at which the separation is particularly uncomplicated and in particular non-cryogenic. If it is stated below that ethane or another paraffin other than methane is recycled into the process, this can in particular mean a recirculation into the post-catalytic steam cracking. Methane which is “recycled into the process”, on the other hand, is supplied in particular to the oxidative coupling of methane as feed. However, recirculation can also take place together and in particular together with carbon monoxide in the oxidative coupling overall.

Hydroformylation is another technology which is used in particular for the production of oxo compounds of the type mentioned at the outset. Propylene is typically converted in the hydroformylation, but higher hydrocarbons, in particular hydrocarbons having six to eleven carbon atoms, can also be used. The conversion of hydrocarbons having four and five carbon atoms is also possible in principle, but is of lower practical impact. Hydrogenation can follow the hydroformylation in which aldehydes can initially be formed. Alcohols formed by such hydrogenation can be subsequently dehydrated to give the respective olefins.

In Green et al., Catal. Lett. 1992, 13, 341, a method for the production of propanal from methane and air is described. In the method presented, low yields based on methane are generally observed. In the method, oxidative coupling of methane (OCM) and partial oxidation of methane (PDX) to hydrogen and carbon monoxide are carried out, which are then followed by hydroformylation. The target product is the aforementioned propanal which has to be isolated as such. A limitation arises from the oxidative coupling of methane to give ethylene, for which, at present, typically only lower conversions and limited selectivities are achieved. Differences of the method described in Green et al. from the present invention are explained below with reference to the advantages that can be achieved according to the invention.

The hydroformylation reaction in the aforementioned method is carried out on a typical catalyst at 115° C. and 1 bar in an organic solvent. The selectivity with respect to the (undesirable) by-product ethane is in the range of approx. 1% to 4%, whereas the selectivity with respect to propanal should achieve more than 95%, typically more than 98%. Extensive integration of process steps or the use of the carbon dioxide formed in large amounts as a by-product, in particular in the oxidative coupling of methane, is not described further here, and so there are thus disadvantages compared with conventional processes. Since partial oxidation is used in the process as a downstream step for oxidative coupling, that is to say there is a sequential interconnection, large amounts of unconverted methane in the partial oxidation have to be managed or separated off in a complex manner from the oxidative coupling.

U.S. Pat. No. 6,049,011 A describes a method for the hydroformylation of ethylene. The ethylene can in particular be formed from ethane. Besides propanal, propionic acid can also be produced as the target product. Dehydration is also possible. However, this document does not disclose any further integration and does not disclose any meaningful utilization of the carbon dioxide formed.

In the water gas shift reaction (WGSR), carbon monoxide is converted with steam to form carbon dioxide and hydrogen. This is an exothermic equilibrium reaction, wherein, if necessary, hydrogen with carbon dioxide can also be converted to carbon monoxide and water in the opposite direction (reversed water gas shift, RWGS). Details can be found, for example, in the articles “Hydrogen, 2. Production” and “Synthesis Gas” in Ullmann's Encyclopedia of Industrial Chemistry. A distinction is made between the low temperature shift (LTS) and the high temperature shift (HTS) in the water gas shift.

For high-temperature processes, it is possible in particular to use iron oxide or chromium oxide catalysts which are subjected to a feed gas at about 350° C. The result is a rise in temperature to 400 to 450° C. due to the exothermicity of the shift reaction. In order to avoid excessively high exit temperatures, the entry temperature is correspondingly limited. In the case of low-temperature methods, the feed gas temperature is about 220° C., and a carbon dioxide removal is typically provided. For low-temperature methods, typically copper, zinc and aluminum mixed oxides with promoters (for example with traces of potassium) are used.

A commercially available catalyst for a high-temperature method comprises, for example, about 74.2% diiron trioxide, 10.0% dichromium trioxide, 0.2% magnesium oxide and volatile components in the remaining residue. The chromium oxide acts to stabilize the iron oxide and prevents sintering. High-temperature reactors used on an industrial scale operate in a range from atmospheric pressure to about 8 MPa.

The typical composition of a commercial catalyst for a low-temperature process is 32 to 33% copper oxide, 34 to 53% zinc oxide, 15 to 33% dialuminum trioxide. The active catalytic species is copper oxide and the function of the zinc oxide comprises the prevention of the poisoning of copper by sulfur. The dialuminum trioxide prevents dispersion and pellet shrinkage. The upper temperature limit in the case of low-temperature methods results from the susceptibility of copper to thermal sintering. These lower temperatures also reduce the occurrence of side reactions.

In principle, high-temperature and low-temperature methods can be used for the water gas shift in the context of the present invention. These also depend in particular on the existing or usable amounts of heat. For example, in the optionally used oxidative coupling, a large amount of heat of the product mixture formed at high temperatures can be used, for example, for preheating the use in a high-temperature method.

### Advantages of the Invention

Against this background, the present invention proposes a method for preparing a target compound, in particular propylene, in which a first gas mixture is provided which contains at least one olefin having a first carbon number and carbon monoxide. It is provided within the scope of the present invention that methane with oxygen is subjected to an oxidative coupling to obtain ethylene and further components, including the aforementioned carbon monoxide, but also optionally unconverted methane and ethane and carbon dioxide. The first gas mixture represents a starting mixture which is further processed in the context of the present invention for producing the target compound. Depending on the type of provision, the first gas mixture can also contain water. Hydrogen can also be contained in the first gas mixture. However, the presence of hydrogen and other components is not a requirement, even if a corresponding first gas mixture should be described below as containing hydrogen or further components. The oxidative coupling can also be carried out, for example, without the presence or formation of hydrogen. As mentioned several times, it is not necessarily the subject matter of the invention, but is provided in one embodiment.

As already mentioned at the outset, the oxidative coupling of methane is a process which is, in principle, known from the prior art. In the context of the present invention, known method concepts can be used for the oxidative coupling of methane.

In embodiments of the present invention (substantially) pure methane, or natural gas or associated gas fractions of different purification stages right up to corresponding raw gas can be used as a methane supplier. For example, natural gas can also be fractionated, wherein, when an oxidative coupling is used, methane can be conducted into the oxidative coupling itself and higher hydrocarbons preferably into a post-catalytic steam cracking. Oxygen is particularly preferred as an oxidizing agent in a corresponding method. Air or oxygen-enriched air can in principle likewise be used, but lead to nitrogen entry into the system. A separation at a suitable location in the process would in turn be comparatively complicated and would have to be carried out cryogenically.

In the oxidative coupling, in the present invention, in which it is used, a diluent medium, preferably steam, but also for example carbon dioxide, can be used, in particular for the moderation of the reaction temperatures. Carbon dioxide can also serve (partially) as an oxidizing agent. In principle, compounds suitable as diluents, such as nitrogen, argon and helium, in turn require a complex separation. However, in the current state of the technology, recycled methane serves as diluent, which is converted only to a relatively small proportion.

In embodiments of the present invention, the oxidative coupling can be carried out in particular at an overpressure of 0 to 30 bar, preferably 0.5 to 5 bar, and a temperature of 500 to 1100° C., preferably 550 to 950° C. In principle, catalysts known from technical literature can be used, see, for example, Keller and Bhasin, J. Catal. 1982, 73, 9, Hinsen and Baerns, Chem. Ztg. 1983, 107, 223, Kondrenko et al., Catal. Sci. Technol. 2017, 7, 366-381. Farrell et al., ACS Catalysis 6, 2016, 7, 4340, Labinger, Catal. Lett. 1, 1988, 371, as well as Wang et al., Catalysis Today 2017, 285, 147.

In the context of the present invention, the conversion of methane in the oxidative coupling can be in particular more than 10%, preferably more than 20%, particularly preferably more than 30% and in particular up to 60% or 80%. The particular advantage of the present invention, in which an oxidative coupling is used, is, however, not primarily in the increased yield, but that in particular, in addition, a relatively high relative proportion of carbon monoxide with respect to ethylene in the product mixture of the oxidative coupling, i.e. the first gas mixture used in the context of the present invention, can also be utilized, and that these can be operated in an optimized manner by the use of a water gas shift, as explained below.

Typical by-products of the oxidative coupling of methane are carbon monoxide and carbon dioxide formed in the low to two-digit percentage range. A typical product mixture of the oxidative coupling of methane within the meaning of the invention has, for example, the following mixture ratios:

Hydrogen 0.1 to 10 mole percent

Methane 20 to 90 mole percent

Ethane 0.5 to 30 mole percent

Ethylene 5 to 50 mole percent

Carbon monoxide 5 to 50 mole percent

Carbon dioxide 0.5 to 30 mole percent

These figures refer to the dry fraction of the product mixture, which can also comprise in particular water vapor. Further components, such as higher hydrocarbons and aromatics, can be present in concentrations of typically less than 5 mole percent, in particular less than 1 mole percent, oxigenates—i.e. aldehydes, ketones, ethers, etc.—may be present in traces, i.e. typically less than 0.5 mole percent, in particular less than 0.1 mole percent, in the product mixture of the oxidative coupling.

As already mentioned several times, a first gas mixture provided in a non-inventive embodiment can also be formed by other methods, or other methods can be involved in the formation thereof. The composition of the gas mixture can in particular be as described above for the product mixture of the oxidative coupling, but also differ therefrom.

In the context of the present invention, a second gas mixture which is formed using at least a portion of the first gas mixture and which contains at least the olefin having the first carbon number, hydrogen and carbon monoxide, to obtain a third gas mixture containing a compound having a second carbon number and at least carbon monoxide, is subjected to one or more conversion steps which comprises or comprise a hydroformylation. Both the first and the second gas mixture can also contain carbon dioxide. Carbon dioxide can be formed in particular in the case of an oxidative coupling of methane, but also originate from other methods and in this way reach the first and/or second gas mixture. For example, carbon dioxide is also formed in the water gas mixture according to the invention. The formation of the second gas mixture using at least a portion of the first gas mixture can also comprise, in particular, the removal of carbon dioxide from the first gas mixture or a part thereof, wherein the remaining residue is used partly or completely to form the second gas mixture. A separation of carbon dioxide can also take place at a suitable point further downstream. As explained below, the formation of the second gas mixture always also comprises the addition of hydrogen from a water gas shift used according to the invention.

Since the oxidative coupling is used, the first gas mixture further contains unconverted methane and/or ethane and/or higher hydrocarbons, in particular paraffins. Hydrogen can also be present. Besides the carbon monoxide, the third gas mixture can also contain further components, in particular secondary compounds, which are formed in the one or more conversion steps. Compounds, for example paraffins, such as methane and/or ethane, can also pass into the third gas mixture from the first gas mixture without conversion in the one or more conversion steps.

The second carbon number is one greater than the first carbon number due to the hydroformylation reaction which is part of the one or more conversion steps. Since according to the invention, the oxidative coupling of methane is used, the olefin with the first carbon number is ethylene and, in the case of the compound with the second carbon number, is in particular propanal, propanol and/or propylene.

As explained below, a total of two, three (or more) conversion steps can be provided, comprising the hydroformylation and subsequently a hydrogenation and optionally additionally a dehydration. In each of these steps, a compound having the second carbon number (for example with three carbon atoms) is formed, specifically in the hydroformylation in the form of an aldehyde (for example propanal), in the hydrogenation in the form of an alcohol (for example propanol) from the aldehyde, and in the dehydration in the form of an olefin (for example propylene) from the alcohol. The third gas mixture can thus be a product mixture of each of these conversion steps, i.e. a product mixture from the hydroformylation, a product mixture from the hydrogenation or a product mixture from the dehydration, when several conversion steps are used. It is not ruled out in each case that following the formation of the third product mixture, more of the conversion steps are subsequently carried out, or that only the mentioned conversion steps and not any further conversion steps or other processing steps, such as cleaning, separation, drying or the like, are carried out.

Processes for hydroformylation are also known in principle from the prior art. In recent times, in corresponding processes, as described in the literature cited below, Rh-based catalysts are typically being used. Older methods also employ Co-based catalysts.

For example, homogeneous, Rh(I)-based catalysts with phosphine and/or phosphite ligands can be used. These may be monodentate or bidentate complexes. For the production of propanal, reaction temperatures of 80 to 150° C. and corresponding catalysts are typically used. All methods known from the prior art can also be used in the context of the present invention.

The hydroformylation typically operates at a hydrogen to carbon monoxide ratio of 1:1. However, this ratio can be, in principle, in the range from 0.5:1 to 10:1. The Rh-based catalysts used may have an Rh content of from 0.01 to 1.00% by weight, wherein the ligands may be present in excess. Further details are described in the article “Propanal” in Ullmann's Encyclopedia of Industrial Chemistry, ed. 2012. The invention is not limited by the cited process conditions.

In a further method, as described, for example, in the chapter “Hydroformylation” in Moulijn, Makee & van Diepen, Chemical Process Technology, 2012, 235, a pressure of 20 to 50 bar is used with an Rh-based catalyst and a pressure of 70 to 200 bar is used with a Co-based catalyst. Co also appears to be relevant in metallic form for hydroformylation. Other metals are more or less insignificant, especially Ru, Mn and Fe. The temperature range used in said method is between 370 K and 440 K.

In the method disclosed in the chapter “Synthesis involving Carbon Monoxide” in Weissermel & Arpe, Industrial Organic Chemistry 2003, 135, mainly Co- and Rh-phosphine complexes are used. With specific ligands, hydroformylation can be carried out in aqueous medium and recovery of the catalyst is readily possible.

According to Navid et al., Appl. Catal. A 2014, 469, 357, in principle all transition metals capable of forming carbonyls can be used as potential hydroformulation catalysts, wherein an activity as per Rh>Co>Ir, Ru>Os>Pt>Pd>Fe>Ni is observed according to this publication.

By-products in the hydroformylation are formed in particular by the hydrogenation of the olefin to give the corresponding paraffin, i.e., for example, from ethylene to ethane, or the hydrogenation of the aldehyde to give the alcohol, i.e. from propanal to propanol. According to the article “Propanols” in Ullmann's Enyclopedia of Industrial Chemistry, ed. 2012, propanal formed by hydroformylation can be used as the main source of 1-propanol in industry. In a second step, propanal can be hydrogenated to give 1-propanol.

In general, irrespective of the specific nature, sequence and number of the conversion steps mentioned, in the context of the present invention, at least using at least a portion of the third gas mixture, a fourth gas mixture depleted in the compound with the second carbon number compared to the third gas mixture and enriched in carbon monoxide is formed. This formation of the fourth gas mixture can comprise, in particular, non-cryogenic separation of the compound with the second carbon number, so that more light-boiling compounds remain in the fourth gas mixture. Such separation is particularly simple in particular in the case of the preparation of an aldehyde or alcohol as a compound with the second carbon number due to the comparatively high boiling point. The removal of a corresponding olefin with the second carbon number, for example of propylene, of lower-boiling compounds is likewise comparatively simple in terms of separation technology. Depending on the composition of the third gas mixture, the fourth gas mixture can therefore comprise in particular hydrogen, optionally carbon dioxide, methane, ethane and optionally residues of ethylene. Further light-boiling compounds which are formed in the one or more conversion steps, for example as by-products, can likewise be present. In addition to the compound with the second carbon number, further compounds having the second carbon number and higher-boiling compounds remain in a corresponding residue if they are formed.

If it is stated here that liquids or gases or corresponding mixtures are rich or poor with regard to one or more components, “rich” is intended to mean a content of at least 90%, 95%, 99%, 99.5%, 99.9%, 99.99% or 99.999% and “poor” for a content of at most 10%, 5%, 1%, 0.1%, 0.01% or 0.001% on a molar, weight or volume basis. The term “predominantly” refers to a content of at least 50%, 60%, 70%, 80% or 90% or corresponds to the term “rich”. In the terminology used here, liquids and gases or corresponding mixtures can also be enriched or depleted in one or more components, wherein these terms refer to a corresponding content in a starting mixture. The liquid or the gas or the mixture is “enriched” when at least 1.1 times, 1.5 times, 2 times, 5 times, 10 times, 100 times or 1000 times the content is present, and is “depleted” if at most 0.9 times, 0.5 times, 0.1 times, 0.01 times or 0.001 times the content of a corresponding component, based on the starting mixture, is present. A (theoretically possible) complete separation in this sense represents a depletion to zero with respect to a component in a fraction of a starting mixture, which therefore merges completely into the other fraction and is present there enriched. This is also covered by the terms “enriching” and “depleting”.

If it is stated here that a separation takes place, a mere enrichment of certain substance flows with respect to corresponding components or a depletion with respect to other components can take place at any time. All technologies known to the person skilled in the art can be used here, for example absorptive or adsorptive processes, membrane methods and enrichment or separation steps based on organometallic frameworks.

As mentioned, in embodiments of the present invention, a hydrogenation and optionally a dehydration and/or further conversion steps of the components formed in the hydroformylation, in this case the aldehyde, can also occur for the production of further products. Each of these products can represent a target connection of the method proposed according to the invention.

Hydrogenation of different unsaturated components is a well known and established technology for converting components having a double bond into the corresponding saturated compounds. Typically, very high or complete conversions with selectivities of well above 90% can be achieved. Typical catalysts for the hydrogenation of carbonyl compounds are based on Ni, as is also described, for example, in the article “Hydrogenation and Dehydrogenation” in Ullmann's Encyclopedia of Industrial Chemistry, ed. 2012. Noble metal catalysts can also be used specifically for olefinic components. Hydrogenations are part of the standard reactions of technical chemistry, as also shown, for example, in M. Baerns et al., “Beispiel 11.6.1: Hydrierung von Doppelbindungen” [“Example 11.6.1: hydrogenation of double bonds”], Technische Chemie 2006, 439. In addition to unsaturated compounds (understood here are olefins, in particular), the authors also mention other groups of substances, such as, for example, aldehydes and ketones in particular as substrates for hydrogenation. Low-boiling substances, such as butyraldehyde from the hydroformylation, are hydrogenated in the gas phase. Here, Ni and certain noble metals, such as Pt and Pd, typically in supported form, are used as hydrogenation catalysts.

For example, in the article “Propanols” in Ullmann's Encyclopedia of Industrial Chemistry, ed. 2012, a heterogeneous gas phase process is described which is carried out at 110 to 150° C. and a pressure of 0.14 to 1.0 MPa at a hydrogen to propanal ratio of 20:1. Reduction takes place with excess hydrogen and the heat of the reaction is dissipated by circulating the gas phases through external heat exchangers or by cooling the reactor in the interior. The efficiency with respect to hydrogen is more than 90%, the conversion of the aldehyde is effected up to 99.9% and alcohol yields of more than 99% result. Widely used commercial catalysts include combinations of Cu, Zn, Ni and Cr supported on aluminum oxide or kieselguhr. Dipropyl ether, ethane and propyl propionate are mentioned as typical by-products which can form in traces. According to the general prior art, the hydrogenation is preferably effected in particular only with stoichiometric amounts of hydrogen or only a low hydrogen excess.

Details of corresponding liquid-phase processes are also given in the literature. These are carried out, for example, at a temperature of 95 to 120° C. and a pressure of 3.5 MPa. Typically, Ni, Cu, Raney nickel or supported Ni catalysts reinforced with Mo, Mn and Na are preferred as catalysts. 1-propanol can be prepared with 99.9% purity, for example. The main problem with the purification of 1-propanol is the removal of water from the product. If, as in one embodiment of the present invention, propanol is dehydrated to give propylene, water is also one of the reaction products in this step, so that water does not have to be removed beforehand. The separation of propylene and water is thus made simple.

Dehydration of alcohols on suitable catalysts to prepare the corresponding olefins is also known. In particular, the production of ethylene (from ethanol) is common and is gaining importance in connection with the increasing production quantities of (bio)ethanol. Commercial use has been achieved by different companies. For example, reference is made to the aforementioned article “Propanols” in Ullmann's Encyclopedia of Industrial Chemistry and Intratec Solutions' “Ethylene Production via Ethanol Dehydration”, Chemical Engineering 120, 2013, 29. Accordingly, the dehydration of 1- or 2-propanol to give propene has no practical value until now. Nevertheless, the dehydration of 2-propanol in the presence of mineral acid catalysts at room temperature or above is very easy to carry out. The reaction itself is endothermic and equilibrium limited. High conversions are favored by low pressures and high temperatures. Typically, heterogeneous catalysts based on Al₂O₃ or SiO₂ are used. In general, several types of acid catalysts are suitable and, for example, molecular sieves and zeolites can also be used. Typical temperatures range from 200 to 250° C. for the dehydration of ethanol or 300 to 400° C. for the dehydration of 2-propanol or butanol. Owing to the equilibrium limiting, the product stream is typically separated off (separation of the olefin product and also at least partially of the water, for example by distillation) and the stream containing unconverted alcohol is recycled to the reactor inlet. In this way, overall very high selectivities and yields can be achieved.

In the context of the present invention, carbon monoxide in at least a portion of the fourth gas mixture is subjected to a water gas shift to form hydrogen and carbon dioxide. In this case, a separation or enrichment can be conducted upstream of this water gas shift, as is also explained below. Hydrogen formed in the water gas shift is used according to the invention at least in part in the formation of the second gas mixture, and is thus fed to the one or at least one of the plurality of conversion steps.

Overall, the present invention thus proposes (at least) the coupling of a hydroformylation process and a water gas shift, wherein the hydroformylation and optionally subsequent process steps is or are fed with hydrogen, which is formed in the water gas shift, wherein the water gas shift is fed with carbon monoxide from downstream of the hydroformylation, i.e. from the third or via the fourth gas mixture. The invention comprises the provision of the first gas mixture by an oxidative coupling of methane.

In the context of the present invention, particular advantages result from the fact that hydrogen can be provided as required by the water gas shift with the carbon monoxide from the third or fourth gas mixture as the starting material. This is a major aspect of the present invention. In the oxidative coupling of methane, in which carbon monoxide and carbon dioxide are inevitably formed as by-products, it is not necessary to consider the formation thereof due to the adjustability of these components as a result of the water gas shift, but rather the oxidative coupling can be operated under yield-optimized conditions. Therefore, the present invention is particularly advantageous.

In general, a particular advantage in the context of the present invention is also that components from the first or second gas mixture can be used in the hydroformylation and optionally subsequent conversion steps without complex cryogenic separation steps. In particular, optionally formed and/or present paraffins and any methane present from the first or second gas mixture can be carried along in the hydroformylation and then be removed more easily therefrom, or hydrogen, which is optionally contained in the first or second gas mixture, can be used in addition to the hydrogen from the water gas shift for later hydrogenation steps. Paraffins and methane can in this way be easily recycled and reused in a reaction feed, as already explained above with reference to oxidative coupling and post-catalytic steam cracking. Carbon dioxide can be separated from the first gas mixture or a part thereof and obtained in any purity. As mentioned, however, separation is also possible further downstream, i.e., for example, from the second or third gas mixture or in each case a part thereof. Target components from the third gas mixture or subsequent mixtures thereof can conversely be easily separated from the above-mentioned lower-boiling compounds due to the comparatively high boiling points.

In one embodiment, the present invention can also comprise that the carbon dioxide, which has been separated off from the first gas mixture or further downstream and had previously been formed as a by-product during the oxidative coupling, is converted in any process step required, for example also a dry reforming. In dry reforming, corresponding carbon dioxide is converted at least in part with methane to obtain carbon monoxide and/or hydrogen.

By using the water gas shift, the present invention enables a precise adaptation of the respective hydrogen and/or carbon monoxide contents to the respective need for corresponding components in the hydroformylation or the downstream process steps, such as hydrogenation.

The present invention enables an increase in the possible yield of valuable products of the oxidative coupling by the use of the carbon monoxide as a reaction partner in the hydroformylation and in the water gas shift. At the same time, in the context of the present invention, the effort involved in product purification and splitting is reduced, in particular through the avoidance of cryogenic separation steps. The separation in particular of C2 and C3 components can take place at comparatively moderate temperatures and optionally avoiding drying. Overall, the energy efficiency is improved and large circuits which are conventionally required due to the limited conversions in the oxidative coupling are avoided or minimized. Non-value-added steps, such as methanation, for example, are avoided in the context of the present invention, as is the formation of by-products and co-products as in other processes for the production of target products, such as propylene, for example.

The above-mentioned article by Green et al. already describes the synthesis of propanal from methane and air, wherein a low yield overall based on methane is reported. In this method, a linking of oxidative methane coupling and partial oxidation is used, followed simply by a hydroformylation. The target product is propanal, which must be isolated as such. Here, the limitations are the oxidative coupling of methane to ethylene, for which even today only small conversions and limited selectivities are achieved. A further integration of process steps is not described in Green et al. The advantages that can be achieved according to the invention are thus not given here. A scheme cited in Green et al. describes the partial oxidation as a downstream unit for oxidative coupling. Due to this sequential interconnection, large amounts of methane must therefore be dealt with in the partial oxidation, which are not converted in the oxidative coupling. The present invention overcomes corresponding disadvantages by means of the proposed measures.

In Green et al., a water-gas shift is not mentioned at any point; instead, only a recirculation of carbon dioxide in an overall recycling process for partial oxidation is indicated. It is proposed here to separate ethylene, carbon dioxide and water from a product stream in a cryogenic manner, so that a residue containing methane, carbon monoxide and hydrogen remains. This is not feasible in practice, since in the case of a cryogenic separation of carbon dioxide and/or water, a very rapid displacement occurs due to solid carbon dioxide or ice.

In addition to a lack of statements relating to a water gas shift, there are also no statements regarding a corresponding carbon monoxide recycling process in Green et al. Only one carbon monoxide recycling process via a partial oxidation into the inlet of the hydroformylation is outlined.

As mentioned, further by-products can be formed in a method for providing the starting gas mixture, i.e. in accordance with the invention in the oxidative coupling. These can be separated off if appropriately suitable, for example together with reaction water, optionally by condensation and/or water scrubbing from a corresponding product mixture of the oxidative coupling and thus the first gas mixture. Owing to its strong interaction with suitable solvents or washing liquids, carbon dioxide can likewise be removed comparatively easily from the product mixture, wherein it is possible to use known methods for removing carbon dioxide, in particular corresponding scrubbing (for example amine scrubbing). Cryogenic separation is not required, so that the entire method of the present invention, at least including hydroformylation, forgoes cryogenic separation steps. Should subsequent steps require the absence of, or only a very low residual concentration of, carbon dioxide (for example due to catalytic inhibition or poisoning), the residual carbon dioxide content after amine scrubbing can be further reduced by an optional caustic scrubbing as fine cleaning, as required.

Any water-containing gas mixtures occurring in the context of the present invention can be subjected to drying at a suitable point in each case. For example, drying can take place downstream of the hydroformylation if, in one embodiment of the present invention, this takes place in the aqueous phase and the hydrogenation downstream of the hydroformylation requires a dry stream as reaction feed. If this is not necessary for the subsequent process steps, drying does not have to take place until complete dryness; rather, water contents can optionally also remain in corresponding gas mixtures, as long as these are tolerable. Different drying steps can also be provided at different points in the method and optionally with different degrees of drying.

The separation of the aforementioned by-products advantageously takes place completely non-cryogenically and is therefore extremely simple in terms of apparatus and in terms of energy expenditure. This represents a substantial advantage of the present invention over prior art methods which typically require complex separation of components that are undesirable in subsequent process steps.

“Non-cryogenic” separation should be understood to mean a separation or separation step which is carried out in particular at a temperature level above 0° C., in particular at typical cooling water temperatures of from 5 to 40° C., in particular from 5 to 25° C., optionally also above ambient temperature. In particular, however, non-cryogenic separation in the sense referred to here represents a separation without the use of a C2 and/or C3 cooling circuit and it is therefore carried out above −30° C., in particular above −20° C.

In a corresponding first gas mixture originating from an oxidative coupling, typically in addition to the olefin, unconverted methane, ethane and carbon monoxide are present as components. However, corresponding components can also originate from other methods, as mentioned. These compounds can be transferred into the subsequent hydroformylation without difficulty. Paraffins, such as methane and ethane, are typically not converted in the hydroformylation. Since heavier compounds with a higher boiling point or other polarity are formed in the hydroformylation, they can be separated off comparatively easily, and likewise non-cryogenically, from the remaining components with lower boiling points. Instead of complete separation, it is also possible to achieve an enrichment of certain substance flows in corresponding components or a depletion of other components. All technologies known to the person skilled in the art can be used here, for example absorptive or adsorptive processes, membrane methods and enrichment or separation steps based on organometallic frameworks. As mentioned, at least the carbon monoxide is converted therefrom in the water gas shift and hydrogen formed can be fed to the hydroformylation or subsequent conversion steps, such as the hydrogenation.

In particular, methane and ethane, or more generally one or more paraffins, can be recycled into the process in embodiments of the invention, for example in the oxidative coupling used at the stated points, or also into other process steps. Ethane does not necessarily need to be recycled into a separate reactor section for post-catalytic steam cracking, but instead can also be recirculated to the oxidative coupling by the methane without separation. Beforehand, however, in particular a separation into a carbon monoxide fraction and a fraction containing methane and ethane or the one or more paraffins takes place.

In general, the fourth gas mixture contains, in particular, one or more paraffins, a fifth gas mixture being formed in a separation using at least a portion of the fourth gas mixture, which mixture is depleted of the one or more paraffins compared to the fourth gas mixture and enriched in carbon monoxide, the fifth gas mixture being supplied at least in part to the water gas shift.

In detail, the fourth gas mixture can thus in particular contain methane and one or a plurality of further paraffins, wherein the carbon monoxide in at least a portion of the fourth gas mixture is subjected thereby to the water gas shift in that it is transferred to a subsequent fraction and only the latter is subjected to the water gas shift. For instance, the fourth gas mixture contains, in particular, methane and one or more further paraffins, wherein in a separation using at least a portion of the fourth gas mixture a fifth gas mixture is formed, which in relation to the fourth gas mixture is depleted of methane and the at least one paraffin and is enriched in carbon monoxide, and the fifth gas mixture is at least in part fed to the water gas shift. As mentioned, the term “separation” may also comprise a formation of corresponding fractions without complete separation.

In the separation in which the fifth gas mixture is formed, a sixth gas mixture is advantageously also formed which is enriched with respect to the fourth gas mixture with respect to the one or more paraffins and is depleted of carbon monoxide, wherein at least a portion of the sixth gas mixture is used in the provision of the first gas mixture. For example, this sixth gas mixture can be subjected at least in part to the oxidative coupling of methane and/or a further process step used for providing the first gas mixture, in particular the post-catalytic steam cracking.

When such a separation is used, when the sixth gas mixture contains methane, using at least a portion of the sixth gas mixture, a first, methane-containing fraction and a second fraction containing the one or more paraffins can be formed, wherein the first fraction is subjected at least in part to the oxidative coupling of methane and the second fraction at least in part to a post-catalytic steam cracking step downstream of the oxidative coupling of methane. The corresponding fractions are each advantageously substantially free from the other compounds.

In a particularly advantageous development, the present invention can comprise energy integration, that is to say a coupling of heat flows for endo- and exothermic reactions. Exothermic reactions are in particular oxidative coupling and hydroformylation. The water gas shift is also an exothermic reaction. In contrast, endothermic reactions constitute a reforming process which may be provided for the provision of additional hydrogen and the dehydration. In the present invention, in which an oxidative coupling is carried out, the use of the waste heat from this process is advantageous for other methods, since this takes place at a comparatively high temperature level of typically more than 800° C.

In the context of the present invention, the aldehyde formed in the hydroformylation can be the target compound or, in the context of the present invention, this aldehyde can be further converted to give an actually desired target compound. The latter variant in particular represents a particularly preferred embodiment of the present invention.

In particular, when the aldehyde is converted to give the target compound, the aldehyde can first be hydrogenated to give an alcohol which has a carbon chain having the second carbon number, i.e. the same carbon number as the aldehyde. A corresponding method variant is particularly advantageous because for said variant, it is possible to use hydrogen which is formed in the method itself, which can be already present in a feed mixture upstream of the hydroformylation and can be passed through the hydroformylation.

Hydrogen can be fed in at any suitable point in the method according to the invention and its embodiments, in particular upstream of the optionally provided hydrogenation. In this way, hydrogen is available for this hydrogenation. The feeding need not take place directly upstream of the hydrogenation; rather, hydrogen can also be fed in by method or separation steps present or carried out upstream of the hydrogenation. Hydrogen can also be present, for example, in the first gas mixture, and at least a portion of this hydrogen can be used in the hydrogenation. Hydrogen can furthermore also be separated off, however, from a partial stream of a product stream of the water gas shift, or formed as a corresponding partial stream, for example by separation steps known per se, such as pressure swing adsorption.

In a further embodiment of the present invention, during the conversion of the aldehyde to give the actual target compound of the method according to the invention, a dehydration of the alcohol formed by the hydrogenation to give a further olefin (based on the previous olefin contained in the starting gas mixture) takes place, wherein the further olefin, in particular propylene, has a carbon chain with the mentioned second carbon number, i.e. the carbon number of the aldehyde formed beforehand and the alcohol formed therefrom.

In particular, the alcohol formed in the conversion of the aldehyde can be separated off comparatively easily from unconverted paraffin. In this way, a recycle stream of the paraffin can also be formed non-cryogenically here and recycled, for example, into the oxidative coupling.

In a particularly preferred embodiment, the present invention permits the use of all components of natural gas. For this purpose, any natural gas fractions or crude gas can be used, as already explained above for the oxidative coupling of methane. Thus, using natural gas, a methane-containing natural gas fraction and a natural gas fraction containing ethane can be formed, wherein the methane-containing natural gas fraction of the oxidative coupling of methane and the natural gas fraction containing ethane are preferably subjected to the post-catalytic steam cracking step.

Further aspects of the present invention have also already been mentioned in principle. In particular, the carbon dioxide can at least partly be separated from the first gas mixture or downstream thereof and used in some other way and purified, for example. The carbon monoxide and the olefin in the remaining residue of the starting gas mixture and optionally further components therein can be subjected to the hydroformylation at least in part without a prior separation from one another. More generally, the olefin with the first carbon number and the carbon monoxide from the first gas mixture can therefore be subjected at least in part to the hydroformylation in the second gas mixture. As mentioned, in the context of the present invention, in principle a complete non-cryogenic separation of obtained gas mixtures can be achieved. This is not necessarily the case for the separation of natural gas into the methane fraction and the fraction with heavier hydrocarbons mentioned above.

As already mentioned, the starting gas mixture can in particular contain methane and a paraffin, wherein at least a portion of the methane and the paraffin can pass through the hydroformylation unconverted. As mentioned in detail above, this part can be separated off downstream of the hydroformylation and recycled. The separation can take place depending on expediency directly downstream of the hydroformylation, i.e. before each process step following the hydroformylation, or downstream of a process step following the hydroformylation, for example after hydrogenation or dehydration, but also after any separation or work-up steps.

In the context of the present invention, as already mentioned, a hydrogen quantity formed in the water gas sulfite can be adapted to a hydrogen requirement in the hydroformylation and/or hydrogenation. Precisely here, there is a particular advantage of the present invention.

In the present invention, the first gas mixture, in particular after a condensate removal taking place during the provision of the first gas mixture, is compressed to a pressure level at which the hydroformylation is carried out and optionally the carbon dioxide is separated off. Additional intermediate steps can also be provided between the optionally provided removal of carbon dioxide and the hydroformylation. In one embodiment of the present invention, the water gas shift is carried out at a lower pressure level, so that the pressure level of the hydroformylation and optionally the carbon dioxide removal represents the highest of the pressure levels. In this way, a further compression can be dispensed with. The provision of the first gas mixture for which the oxidative coupling is used, advantageously at the pressure level previously indicated for the oxidative coupling of methane and the hydroformylation is advantageously carried out at a pressure level of 15 to 100 bar, in particular 20 to 50 bar.

The present invention can be implemented by means of an installation for producing a target compound, in relation to which reference is expressly made to the corresponding independent patent claim. A corresponding installation, which is preferably set up for carrying out a method, as has been explained above in different embodiments, benefits in the same way from the advantages already mentioned above.

The installation has a reactor arrangement configured to provide the first gas mixture using an oxidative coupling of methane.

The invention will be explained in more detail below, initially with reference to the accompanying drawing, which illustrates a preferred embodiment of the present invention. An exemplary embodiment of the invention is subsequently explained in more detail, which is carried out in particular using the method illustrated in the drawing.

### BRIEF DESCRIPTION OF THE DRAWINGS

FIG. 1 illustrates a method according to one embodiment of the invention in the form of a schematic flowchart.

If reference is made below to process steps, such as the oxidative coupling of methane, the water gas shift or hydroformylation, these are also to be understood to cover the apparatus used in each case for these process steps (in particular, for example, reactors, columns, scrubbing devices, etc.), even if reference is not expressly made thereto. In general, the explanations relating to the method apply to a corresponding installation in the same way in each case.

The invention is described below using the inventive example of the oxidative coupling to provide the first gas mixture. This requires carbon dioxide separation.

### DETAILED DESCRIPTION OF THE DRAWINGS

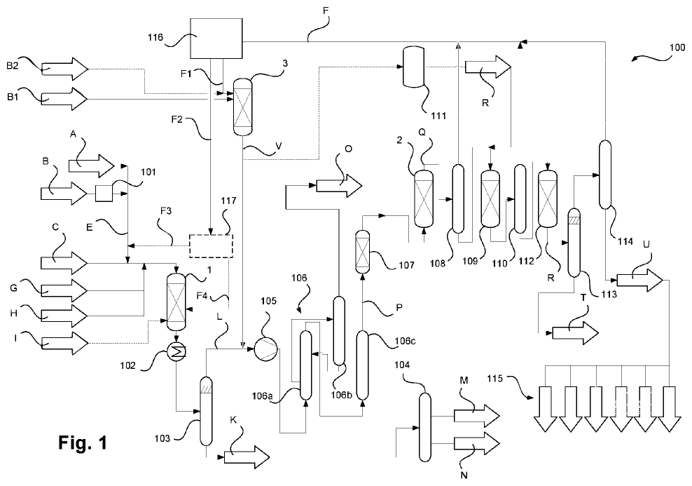

FIG. 1 illustrates a method according to a particularly preferred embodiment of the present invention in the form of a schematic flowchart and is designated overall by 100.

Central process steps or components of the method 100 are an oxidative coupling of methane, which is designated here overall by 1, and a hydroformylation, which is designated here overall by 2. The method 100 further comprises a water gas shift, designated here overall by 3.

In the example shown, a methane stream A is fed to the method 100 or the oxidative coupling of methane 1. Instead of the methane stream A or in addition to this, a raw natural gas stream B can also be provided. If necessary, the raw natural gas stream B can be prepared by means of any treatment step 101. A correspondingly provided input current is denoted by E for better differentiation. Furthermore, in the example illustrated here, a vapor stream B1 and (optionally) a material stream B2 containing water and/or carbon monoxide are provided from an external source in the example illustrated here.

The feed stream E, together with a partial stream, designated here by F3, of a recycle stream F (or, as explained below, optionally also together with a recycle stream F2 comprising further components), is fed to the oxidative coupling 1. In this case, mixing with oxygen, which is provided in the form of a material stream C, and optionally with vapor, which is provided in the form of a material stream G, is carried out. The vapor of the material stream G, like nitrogen of an optionally provided nitrogen stream H, serves as a diluent or moderator and in this way prevents in particular a thermal runaway in the oxidative coupling 1. Water can also make a contribution in order to ensure the catalyst stability (long-term performance) and/or to enable a moderation of the catalyst selectivity.

A reactor used in the oxidative coupling 1 can have a region for performing a post-catalytic steam cracking, as was explained at the outset. A partial stream F4 of the recycle stream F containing ethane can optionally be fed into this region. Alternatively or additionally, it is also possible to feed a separately provided ethane stream I. A feed of propane can also be provided in principle. The ethane stream I and optionally propane and heavier components can also be separated from raw natural gas, the remainder of which is then provided as methane stream A.

Downstream of the oxidative coupling, an aftercooler 102 is provided downstream of which there is, in turn, a condensate separation 103. A condensate stream K formed in the condensate separation 103, which predominantly or exclusively contains water and optionally further, heavier compounds, can be fed to a facility 104 in which, in particular, a (purified) water stream M and residual stream N can be formed.

The product mixture of the oxidative coupling 1 freed from condensate, which is referred to here generally as the first gas mixture is combined in the form of a material stream L with a stream V from the water gas shift 3, which is rich in hydrogen and carbon monoxide and optionally contains carbon monoxide and/or water that is not converted in the water gas shift, and optionally further components, and subsequently compressed in a compressor 105 and subsequently fed to a carbon dioxide removal designated as 106 which can, for example, be carried out using corresponding washes. In the embodiment shown here, a scrubbing column 106a for an amine scrubbing and the regeneration column 106b for the amine-containing scrubbing liquid loaded with carbon dioxide in the scrubbing column 106a are shown. An optional scrubbing column 106c for fine purification, for example for a caustic scrubbing, is also shown. As mentioned, carbon dioxide removal by corresponding scrubbing is generally known. It is therefore not explained separately.

A carbon dioxide stream O formed in the carbon dioxide removal 106 can be fed in particular in purified form to any intended use. It is particularly suitable for subsequent use in further processing methods, since it has a comparatively high concentration of carbon dioxide and a high purity.

A mixture of components remaining in the form of a material stream P, after the removal of carbon dioxide in the carbon dioxide removal 106, and which is here designated generally as the second gas mixture, contains predominantly ethylene, ethane, hydrogen and carbon monoxide. It is optionally dried in a dryer 107 and then fed to the hydroformylation 2.

In the hydroformylation 2, propanal is formed from the olefins and carbon monoxide, which together with the further components explained is carried out in the form of a material stream Q from the hydroformylation 2. In this case, unconverted ethane and further light compounds, such as methane and carbon monoxide, which can be converted into the recycle stream F, can optionally be separated off from the material stream Q in a separation 108. Alternatives to separation 108 are explained further below.

In one of a hydrogenation 109, the propanal can be converted to propanol. The alcohol stream is fed to a further separation 110 optionally provided as an alternative to the separation 108, where components with a lower boiling point can also be separated off and transferred to the recycle stream F.

The hydrogenation 109 can be operated with hydrogen which is contained in a product stream of the water gas shift 3 and is carried along in the hydroformylation. Alternatively, the separate feeding of required hydrogen in the form of a material stream R is also possible, in particular from a separation of hydrogen in a pressure swing adsorption 111.

A product stream from the hydrogenation 109 or from the optionally provided separation 110 is fed to a dehydration 112. In said dehydration, propylene is formed from the propanol. A product stream S from the dehydration 112 is fed to a condensate separation 113 where it is freed of condensible compounds, in particular water. The water can be carried out of the process in the form of a water stream T. The water streams N and T can, optionally after a suitable work-up, also be fed again to the process for steam generation. In this way, for example, at least a part of the steam flow B1 can be provided.

The gaseous residue remaining after the condensate separation 113 is fed to a further separation 114 optionally provided as an alternative to the separations 108 and 110 where, in particular, non-converted ethane and light compounds can also be separated off and transferred to the recycle stream F. A product stream U formed in the separation 114 can be carried out of the process and further process steps, for example for the production of plastics or other further compounds, can be used, as indicated here overall by 115. Corresponding methods are known per se and comprise the use of the propylene from the method 100 as intermediate product or starting product in the petrochemical value chain.

Non-converted ethane and other light compounds, such as methane and carbon dioxide are recycled, as mentioned several times, in the form of a material stream F. For this purpose, in the embodiment illustrated here, a separation 116 is provided, in which a carbon-monoxide-containing or carbon-monoxide-rich partial stream F1, which is also poorer or richer with respect to other components, is formed. Carbon monoxide in this material stream can be converted into the water gas shift 3 to form further hydrogen. The resulting stream V is fed, as described above, at a suitable point before the hydroformylation.

A further partial stream F2 formed in the separation 116, which can in particular contain methane and ethane, is guided into the oxidative coupling 1. In this case, a separation 117 can optionally be provided, in which the partial streams F3 and F4 can be formed, which have already been explained above. In particular, methane and ethane can be separated from one another in this way, wherein the methane in the partial stream F3 in the oxidative coupling 1 can be conducted to the reactor inlet and the ethane in the partial stream F4 can be conducted to a reactor zone used for the post-catalytic steam cracking. However, it is also possible in principle to feed the material stream F2 to the reactor inlet without separation 117.

### Exemplary Embodiment

In the context of the present invention, a starting gas mixture was considered as can be provided according to the invention by means of the oxidative coupling of methane, but which can also originate from other sources. According to the invention, carbon monoxide is typically present in an order of magnitude like the olefin (for example ethylene) or even in stoichiometric excess. However, the hydrogen fraction is not sufficient according to the invention to cover the stoichiometric demand for the hydroformylation and any possible subsequent further conversion—as in the case described here of a hydrogenation.

The following gross equation results for an ideal overall reaction according to one embodiment of the present invention of the proposed integrated process downstream of the provision of the starting gas mixture (hydroformylation, hydrogenation and dehydration):

C₂H₄+2H₂+CO→C₃H₆+H₂O (I)

A targeted and demand-based adjustment of the ratio of hydrogen to carbon monoxide is possible by the use of the water gas shift provided according to the invention as follows:

CO+H₂O→H₂+CO₂ (II)

Other embodiments of the oxidative coupling can also lead in particular to a low or very low hydrogen content in the product gas of the oxidative coupling. Accordingly, there can also be a corresponding disparity in another gas mixture. Here too, in the context of the present invention, the additional provision of hydrogen is, on the one hand, made possible precisely by the above-mentioned water gas shift reaction. A corresponding provision of further additional hydrogen can moreover take place from other sources, for example by means of classical reforming or from water electrolysis.

A calculation example based on the oxidative coupling is given below to document the advantages that can be achieved according to the present invention, in which the component fractions required or advantageous for an starting gas mixture are determined in particular.

The gross equation I indicated above results for an ideal overall reaction of the integrated process after oxidative coupling (hydroformylation, hydrogenation and dehydration).

The carbon monoxide nₜₒₜₐₗ(CO) and hydrogen nₜₒₜₐₗ (H₂) required by the hydroformylation and hydrogenation reaction cascade is 1 mol of carbon monoxide per 1 mol of ethylene and 2 mol of hydrogen per 1 mol of ethylene. The amount of ethylene in the product stream of the oxidative coupling is n_{OCM}(C₂H₄), the amount of carbon monoxide is n_{OCM}(CO) and the amount of hydrogen is n_{OCM}(H₂).

After the oxidative coupling, the process gas preferably contains a high proportion of carbon monoxide and a certain proportion of hydrogen. If the ratio of carbon monoxide to hydrogen according to the stoichiometric demand of equation I is now set by a shift reaction according to equation II above, the amount of hydrogen n_{Shift}(H₂) and simultaneously the same amount of carbon monoxide n_{Shift}(CO) are used:

n_{Shift}(H₂)=n_{Shift}(CO) (III)

Any additional demand for CO and H₂ required is optionally covered from an external source, such as a reforming process. The amounts of substance from this external source are hydrogen nₑₓₜₑᵣₙₐₗ(H₂) and for carbon monoxide nₑₓₜₑᵣₙₐₗ(CO).

Thus, the CO and hydrogen required by gross equation I are covered as follows:

nₜₒₜₐₗ(CO)=n_{OCM}(CO)−n_{Shift}(CO)+nₑₓₜₑᵣₙₐₗ(CO) (IV)

nₜₒₜₐₗ(H₂)=n_{OCM}(H₂)+n_{Shift}(H₂)−nₑₓₜₑᵣₙₐₗ(H₂) (V)

In the shift arrangement, the following amount of hydrogen is thus provided:

n_{Shift}(H₂)=nₜₒₜₐₗ(H₂)−n_{OCM}(H₂)−nₑₓₜₑᵣₙₐₗ(H₂) (VI)

By inserting equation VI in equation IV, taking into account equation III, the CO requirement nₜₒₜₐₗ (CO) results as follows:

nₜₒₜₐₗ(CO)=n_{OCM}(CO)−[nₜₒₜₐₗ(H₂)−n_{OCM}(H₂)−nₑₓₜₑᵣₙₐₗ(H₂)]+nₑₓₜₑᵣₙₐₗ(CO) (VII)

According to the stoichiometry of gross equation I, the following applies under ideal conditions:

nₜₒₜₐₗ(CO)=n_{OCM}(C₂H₄) (VIII)

nₜₒₜₐₗ(H₂)=2n_{OCM}(C₂H₄) (IX)

Following adjustment, the insertion of equation VIII and IX in equation VII results in the following:

3n_{OCM}(C₂H₄)=n_{OCM}(CO)+n_{OCM}(H₂)+nₑₓₜₑᵣₙₐₗ(H₂)+nₑₓₜₑᵣₙₐₗ(CO) (X)

To avoid an external supply of CO and/or H₂, (nₑₓₜₑᵣₙ(H₂)=nₑₓₜₑᵣₙ(CO)=0) therefore, the product gas of the OCM ideally fulfills the following equation:

3n_{OCM}(C₂H₄)=n_{OCM}(CO)+n_{OCM}(H₂) (XI)

In this case, the shift reaction according to equation II reliably represents the required ratio between CO and H₂.

An import of CO and/or H₂ is therefore necessary if the following applies:

3n_{OCM}(C₂H₄)>n_{OCM}(CO)+n_{OCM}(H₂) (XII)

An excess of CO and/or H 2 is present, however, if the following applies:

3n_{OCM}(C₂H₄)<n_{OCM}(CO)+n_{OCM}(H₂) (XIII)

These considerations are based on idealized assumptions, but may help to derive a preferred range for gas compositions. As a result of the integration of the water gas shift provided according to one embodiment of the invention, the ratio of carbon monoxide and hydrogen can be set as required and flexibly.

1-15. (canceled)

16. A method for producing a target compound comprising:
providing first gas mixture comprising an olefin having a first carbon number and carbon monoxide,
forming a second gas mixture comprising at least a portion of the first gas mixture, the olefin having the first carbon number, hydrogen, and carbon monoxide, obtaining a third gas mixture comprising a compound having a second carbon number and carbon monoxide,
subjecting the third gas mixture to one or more conversion steps, wherein the one or more conversion steps comprise a hydroformylation process, and wherein the second carbon number is one greater than the first carbon number,
providing the first gas mixture by using an oxidative coupling of methane, and wherein the first gas mixture comprises ethylene as the olefin having the first carbon number, and methane, ethane and carbon dioxide, and wherein the carbon dioxide is at least partly separated from the first gas mixture or a part of the first gas mixture while leaving the second gas mixture,
forming a fourth gas mixture comprising the third gas mixture, wherein the fourth gas mixture contains less of the compound with the second carbon number than the third gas mixture, and wherein the fourth gas mixture is enriched in carbon monoxide in such a way that the carbon monoxide in at least a portion of the fourth gas mixture is subjected to a water gas shift to form hydrogen and carbon dioxide, and wherein the hydrogen formed in the water gas shift is used at least in part to form the second gas mixture,
compressing the first gas mixture to a first pressure level, wherein the hydroformylation process is carried out at a second pressure level, wherein the water gas shift is carried out at a third pressure level, and wherein the second pressure level is higher than the first and the third pressure levels.

17. The method of claim 16 wherein, the fourth gas mixture comprises one or more paraffins, the method further comprising:
forming a fifth gas mixture in a separation process using at least a portion of the fourth gas mixture, where in the fifth gas mixture has less parafins than the fourth gas mixture and wherein the fifth gas mixture is enriched in carbon monoxide, and
feeding the fifth gas mixture at least in part to the water gas shift.

18. The method of claim 17 further comprising:
forming a sixth gas mixture during the separation process of the fifth gas mixture wherein the sixth gas mixture has more paraffins and fewer carbon monoxide than the fourth gas mixture, and wherein at least a portion of the sixth gas mixture is used when providing the first gas mixture.

19. The method of claim 16 wherein the conversion steps in addition to the hydroformylation process comprise one or more further conversion steps in which the one or more compounds having the second carbon number comprise the aldehyde formed in the hydroformylation process, and wherein one or more further compounds are formed in one or more further subsequent steps.

20. The method of claim 19 further comprising:
forming the fourth gas mixture downstream of the one or more subsequent steps.

21. The method of claim 19 wherein the one or more subsequent steps comprise a hydrogenation process in which the aldehyde is converted with hydrogen to form an alcohol.

22. The method of claim 21 wherein, the first gas mixture contains hydrogen and in which at least a portion of the hydrogen is used in the hydrogenation process.

23. The method of claim 21 wherein, at least one subsequent step comprises a dehydration process converting the alcohol to a second olefin.

24. The method of claim 16 further comprising:
adapting a hydrogen quantity formed in the water gas shift to a hydrogen requirement in at least one of the hydroformylation process or the hydrogenation process or combinations thereof.

25. The method of claim 16 further comprising:
feeding the olefin having the first carbon number and the carbon monoxide from the first gas mixture to the hydroformylation process, and wherein the olefin having the first carbon number and the carbon monoxide from the first gas mixture are at least partially unseparated from each other in the second gas mixture.

26. The method of claim 16, wherein the method is carried out completely non-cryogenically downstream of the water gas shift, and an oxidative dehydrogenation process, or the water gas shift and the oxidative coupling process.
